(19) United States
(12) Patent Application Publication
(10) Pub. No.: US 2021/0253721 A1
(43) Pub. Date: Aug. 19, 2021

(54) NOVEL CANCER TREATMENT INVOLVING MODULATION OF IL-3 ACTIVITY

(71) Applicants: UNIVERSITY OF SOUTH AUSTRALIA, Adelaide (AU); CENTRAL ADELAIDE LOCAL HEALTH NETWORK INC, Adelaide (AU)

(72) Inventors: Angel F. Lopez, Medindie (AU); Claudine S. Bonder, Prospect (AU); Emma Thompson, Rostrevor (AU)

(21) Appl. No.: 17/160,096

(22) Filed: Jan. 27, 2021

Related U.S. Application Data
(63) Continuation of application No. 15/753,614, filed on Feb. 20, 2018, now abandoned.

(30) Foreign Application Priority Data
Aug. 18, 2015 (AU) 2015903329

(51) Int. Cl. C07K 16/28; G01N 33/574; A61P 35/04; C07K 16/24

(52) U.S. Cl. CPC C07K 16/2866; G01N 33/57423; G01N 33/57407; G01N 33/57438; G01N 33/57415; A61K 2039/505; C07K 16/244; G01N 2800/50; G01N 2800/52; G01N 2333/5403; A61P 35/04

(57) ABSTRACT

A method of treating or preventing breast cancer (eg invasive ductal carcinoma) and/or cancer associated with elevated levels of either one or both of the IL-3 receptor (IL-3R) and interleukin-3 (IL-3) is disclosed which comprises administering to a subject an IL-3-inhibiting agent such as an agent which inhibits (eg by blocking) IL-3R.

### TECHNICAL FIELD

The present inventors have identified a single pathogenic factor, IL-3, which promotes the formation of blood vessel structures in breast cancer. This disclosure relates to a method for modulating the activity of IL-3 in a subject suffering from breast cancer and/or cancer associated with elevated levels of either one or both of the IL-3 receptor (IL-3R) and IL-3, preferably through inhibiting (eg blocking) the IL-3R, a heterodimeric receptor comprising an α chain and a β chain. In one particular application, the method involves administering to a subject suffering from invasive ductal carcinoma (including invasive ductal carcinoma which has been assessed as having vascular potential through the detection of an elevated level of either one or both of IL-3R and IL-3), a therapeutically effective amount of an agent which inhibits the activity of IL-3R (eg an anti-IL-3R antibody) in the subject.

### PRIORITY DOCUMENT

The present application claims priority from Australian Provisional Patent Application No 2015903329 titled “Novel cancer treatment” filed on 18 Aug. 2015, the content of which is hereby incorporated by reference in its entirety.

### BACKGROUND

Breast cancer is the most commonly diagnosed cancer among women in Australia and elsewhere and will continue to rise with the ageing population. Breast cancer is a heterogeneous disease with the most prominent predictive and prognostic factors being the expression of hormone receptors (eg oestrogen receptor (ER), progesterone receptor (PR) and human epidermal growth factor receptor (HER2))¹. Breast cancer can also be categorised into different molecular subtypes (eg luminal A, luminal B, and basal-like), and these show different angiogenic characteristics at gene and protein levels with the basal carcinomas having the highest vascular content^{2,3}. Based on stratifications such as these, breast cancer patients receive targeted therapy. Importantly, the triple negative breast cancers (TNBC) represent ˜20% of the breast cancers worldwide, are the most aggressive with a high tendency to metastasise, and do not benefit from endocrine therapy or anti-HER2 antibody treatment²³.

As vascular endothelial growth factor (VEGF) has been shown to have a significant role in the progression and prognosis of many cancers (including breast cancer), it has been targeted as a treatment option. For example, Bevacizumab (Avastin®) is a humanised monoclonal antibody targeting all known isoforms of vascular endothelial growth factor (VEGF)-A, and has quickly become the most widely tested anti-angiogenic treatment in breast cancer clinical trials, particularly for the TNBC ER⁻ PR⁻ HER2⁻ patients^{1,4}. However, adding this drug (well-known as a successful treatment for lung and colorectal cancers) to standard post-surgery therapy for breast cancer has not been found to improve progression-free survival or overall survival over standard therapy alone⁴. In addition, Bevacizumab or Sunitinib have been shown to accelerate cancer metastasis, including breast cancer, together with marked hypoxia and vasculogenic mimicry (VM) formation in mice receiving short-term therapy^{5, 24}.

Cancer progression requires the tumour to access the blood supply for the provision of oxygen and nutrients, and consequently, the presence of a highly vascularised tumour(s) has been found to correlate directly with poor prognosis. Tumour vascularisation can occur via a number of processes; including, the endothelial cell (EC)-dependent processes of angiogenesis (the proliferation of existing blood vessel ECs, which form the inner monolayer of blood vessels) and vasculogenesis (the mobilisation of bone-marrow-derived endothelial progenitor cells (EPCs) into the bloodstream), as well as an EC-independent manner known as vasculogenic mimicry (wherein vascular-like channels are formed by the cancer cells themselves). However, in the case of breast cancer progression, it is believed that the required tumour vascularisation primarily results through the process of vasculogenesis, VM or a combination of both. Consequently, the present inventors considered that the identification of a single pathogenic factor which promotes one or both of these processes could lead to the development of novel therapies and assays (eg assays for diagnosis/prognosis and/or disease stratification) that might lead to, for example, improved disease outcome.

### SUMMARY

In a first aspect, the present disclosure provides a method of treating or preventing breast cancer in a subject, said method comprising administering to said subject an interleukin-3 (IL-3)-inhibiting agent, such as, for example, an anti-IL-3R antibody.

The breast cancer that may be treated or prevented by the method may be a basal-like breast cancer such as the triple negative breast cancer (TNBC), invasive ductal carcinoma (IDC).

The method of the first aspect may further comprise a pre-treatment step comprising determining the breast cancer of the subject as having vascular potential or being VM competent by detecting an elevated level of either one or both of IL-3R and IL-3.

In some embodiments, the method of the first aspect involves the administration of an IL-3-inhibiting agent comprising an anti-IL-3R antibody or IL-3R-binding fragment thereof.

In a second aspect, the present disclosure provides a method of diagnosing or prognosing breast cancer in a subject, said method comprising detecting an elevated level of IL-3R or IL-3-present in a suitable body sample of said subject.

In a third aspect, the present disclosure provides the use of an IL-3-inhibiting agent for the manufacture of a medicament for the therapeutic treatment of breast cancer.

In a fourth aspect, the present disclosure provides the use of an IL-3-inhibiting agent for the treatment of breast cancer.

In a fifth aspect, the present disclosure relates to the use of an inhibitory humanised monoclonal anti-IL-3R antibody, an inhibitory humanised monoclonal anti-IL-3 antibody, an inhibitory fully human monoclonal anti-IL-3R antibody or an inhibitory fully human monoclonal anti-IL-3 antibody for the treatment of invasive ductal carcinoma.

In a sixth aspect, the present disclosure provides a method for the prevention or treatment of metastasis in a subject suffering from breast cancer, said method comprising administering to said subject an IL-3-inhibiting agent.

In a seventh aspect, the present disclosure provides a method for the stratification of breast cancer, said method comprising detecting an elevated level of IL-3R or IL-3 present in a suitable body sample of said subject.

The method of the seventh aspect may provide information to further stratify breast cancers beyond different molecular subtypes (eg luminal A, luminal B, and basal-like) such as whether or not the breast cancer has vascular potential or that the breast cancer cells are VM competent.

In an eighth aspect, the present disclosure provides a method of treating or preventing cancer associated with elevated levels of either one or both of IL-3R and IL-3 in a subject, said method comprising administering to said subject an IL-3-inhibiting agent, such as, for example, an anti-IL-3R antibody.

The cancer that may be treated or prevented by the method may be a cancer such as renal cell carcinoma, brain cancer or lung carcinoma.

The method of the eighth aspect may further comprise a pre-treatment step comprising determining the cancer of the subject as having vascular potential or being VM competent by detecting an elevated level of either one or both of IL-3R and IL-3.

In some embodiments, the method of the eighth aspect involves the administration of an IL-3-inhibiting agent comprising an anti-IL-3R antibody or IL-3R-binding fragment thereof.

In a ninth aspect, the present disclosure provides a method of diagnosing or prognosing cancer associated with elevated levels of either one or both of IL-3R and IL-3 in a subject, said method comprising detecting an elevated level of IL-3R or IL-3-present in a suitable body sample of said subject.

In a tenth aspect, the present disclosure provides the use of an IL-3-inhibiting agent for the manufacture of a medicament for the therapeutic treatment of cancer associated with elevated levels of either one or both of IL-3R and IL-3.

In an eleventh aspect, the present disclosure provides the use of an IL-3-inhibiting agent for the treatment of cancer associated with elevated levels of either one or both of IL-3R and IL-3.

In a twelfth aspect, the present disclosure relates to the use of an inhibitory humanised monoclonal anti-IL-3R antibody, an inhibitory humanised monoclonal anti-IL-3 antibody, an inhibitory fully human monoclonal anti-IL-3R antibody or an inhibitory fully human monoclonal anti-IL-3 antibody for the treatment of renal cell carcinoma, brain cancer or lung carcinoma.

In a thirteenth aspect, the present disclosure provides a method for the prevention or treatment of metastasis in a subject suffering from renal cell carcinoma, brain cancer or lung carcinoma, said method comprising administering to said subject an IL-3-inhibiting agent.

In a fourteenth aspect, the present disclosure provides a method for the stratification of renal cell carcinoma, brain cancer or lung carcinoma, said method comprising detecting an elevated level of IL-3R or IL-3 present in a suitable body sample of said subject.

The method of the fourteenth aspect may provide information to further stratify cancers beyond different molecular subtypes such as whether or not the cancer has vascular potential or that the cancer cells are VM competent.

### BRIEF DESCRIPTION OF DRAWINGS

FIG. 1 provides a schematic diagram of the proposed role of IL-3 in the processes of vasculogenesis and vasculogenic mimicry (VM), wherein in response to tumour growth, endothelial progenitor cells (EPCs) migrate from the bone marrow to the periphery, where they proliferate and differentiate into mature endothelial cells (ECs) for expansion of the local vasculature (vasculogenesis) and/or the formation of channels through vascular mimicry (VM) which can anastomose (fuse) with conventional blood vessels to access the blood supply;

FIGS. 2A-2B provide results showing that VM competent IDC cell lines form VM channels in vitro and in vivo. (A) in vitro Matrigel assays identified IDC cell lines which, like HUVEC, can form tube-like structures (ie VM). 1×10⁴ cells were seeded into 12 μl Matrigel and images captured after 3-6 h. One of n=5. (B) MDA-MB-231 tumours in NOD/SCID mice contain VM (CD31⁻PAS⁺) and EC-lined channels (CD31⁺PAS⁻) as indicated by arrows. H, haematoxylin used to counterstain. One of n=7;

FIG. 3 provides graphical results of IL-3R surface expression on naEPCs, HUVEC, freshly isolated EPCs and MDA-MB-231 cells by fluorescence activated cell sorting (FACS). MDA-MB-231 cells were shown to express EC markers. One of n=3;

FIG. 4 provides results showing IL-3 production by human IDCs. (A-C) in silico analysis of IL-3 and GM-CSF gene expression using the Oncomine database (Compendia Biosciences; Ann Arbor, Mich., United States of America) in IDC breast cancer patients and prostate cancer patients showed that ˜50% of IDC patients exhibited an increase in IL-3 mRNA. (D) IL-3 (dark) stained human IDC but not normal breast tissue;

FIGS. 5A-5C graphically show that basal-like breast cancer patients with high IL-3 gene expression showed decreased overall survival compared to patients with low IL-3 gene expression in a Kaplan-Meier plot from Gene Expression Omnibus (GEO) Datasets documented in Gyorffy et al.²⁵ (FIG. 5A), GSE22220²⁶ (FIG. 5B) and GSE12093+GSE6532 using GOBO²⁷ (FIG. 5C);

FIG. 6 graphically shows that addition of a blocking antibody to the IL-3Rα (7G3) significantly attenuated the VM capability of MDA-MB-231 in an in vitro Matrigel tube formation assay. Untreated (NT), IgG or anti-IL-3Rα mAh (7G3) were added prior to cell seeding in Matrigel. n=5, *p<0.05;

FIG. 7 graphically shows that addition of a blocking antibody to the β-chain of IL-3R (BION-1) attenuated the VM capability of the HS-578-T cell line in an in vitro Matrigel tube formation assay;

FIG. 8 graphically shows the effect of IL-3 augmentation on promoting VM in the HS-578-T cell line;

FIGS. 9A-9C display results of in vivo experiments using modified MDA-MB-231 cells showing a role for IL-3 in breast cancer progression. (A) seven days post-injection of MDA231-LM2 cells, IgG, 7G3 (anti-IL-3Rα mAh) or BION-1 (anti-IL-3Rβ mAh) were added (0.3 mg/kg) every 48 h to the mice. 7G3 or BION-1 attenuated MDA231-LM2 cell tumour development. The human IL-3/GM-CSF expressing transgenic mice exhibited increased tumour growth. n>4, *p<0.05 vs IgG. (B) images of the cancer cell luminescence in vivo are shown. (C) provides tabulated results showing that metastasis was reduced to the lungs, liver, brain and bone marrow by 7G3 or BION-1;

FIGS. 10A-10B display the results of experiments showing that blocking the IL-3 receptor attenuates IDC progression in an in vivo mammary fat pad tumour mouse model. NOD/SCID mice injected with 1×10⁶ MDA-MB-231-LM2 into the mammary fat pad, treated with 0.3 mg/kg of antibodies to (A) IL-3Rα blocking antibody (IL-3Rα), (B) βc blocking antibody (βc), or a control antibody (IgG). Caliper measurements were taken every two days to calculate tumour volume ((width²×length)/2). Dots=individual mice. One-way ANOVA; error bars=mean±SEM (n=7-12), *p=<0.001 vs IgG;

FIGS. 11A-11C provide results of gene expression analysis of vascular markers on MDA-MB-231-LM2 grown in 2D and in vivo compared with vascular cells, showing that when exposed to the tumour microenvironment tumour cells upregulate vascular marker genes. mRNA expression levels for vascular marker genes in MDA-MB-231-LM2 excised from the xenograft tumours (FIG. 1) as well as these cells grown on tissue culture plastic (2D) versus their parental cell line MDA-MB-231 grown on tissue culture plastic (2D) as determined by qPCR with relative gene expression normalised to CycA, GAPDH, and β actin using geNorm software³². Endothelial progenitor cell (EPC) and human umbilical vein endothelial cell (HUVEC) expression levels are also shown for comparison. Error bars: mean±SEM; n=3;

FIGS. 12A-12C show that mammary fat pad xenografts produce human IL-3. Immunohistochemistry of MDA-MB-231-LM2 excised primary tumours DAB-stained for human IL-3. (A) representative images of tumours extracted from mice treated with PBS, 0.3 mg/kg of IgG control antibody (IgG), IL-3Rα blocking antibody (IL-3Rα), or βc blocking antibody (βc). (B) compiled data quantified using online freeware ImmunoRatio. Bars represent mean percentage area stained positive for IL-3 from 10 fov/tumour±SEM; n=5=6. In (C), IL-3 expression normalised to IgG1 isotype control; bars represent mean % area IL-3 positive from 10 fov/tumour±SEM; n=5=6;

FIG. 13 shows that hypoxia increases IL-3 receptor abundance on human breast cancer cell lines. Flow cytometric analysis of IL-3Rα on MDA-MB-231, SUM159, and SUM159-LN2 breast cancer cell lines grown under normal conditions (10% FBS, atmospheric O₂) versus hypoxic conditions (0.5% FBS, 3% O₂) for 24 hours; black histogram=unstained cells, grey-shaded histogram=isotype control, blue histogram=IL-3Rα expression (left panel); and

FIG. 14 shows that hypoxia upregulates gene expression of IL-3Rα and βc. Relative mRNA levels of IL-3Rα and βc in MDA-MB-231, SUM159, and SUM159-LN2 breast cancer cell lines grown under normal conditions (10% FBS, atmospheric O₂) versus hypoxic conditions (0.5% FBS, 3% O₂) for 24 hours as determined by qPCR with relative gene expression normalised to CycA, GAPDH, & βactin using geNorm software, n=1.

### DETAILED DESCRIPTION

The present inventors have identified a single pathogenic factor, IL-3, which promotes the formation of both EC-dependent and EC-independent (ie VM) blood vessel structures in breast cancer.

In a first aspect, the present disclosure provides a method of treating or preventing breast cancer in a subject, said method comprising administering to said subject an interleukin-3 (IL-3)-inhibiting agent such as, for example, an anti-IL-3R antibody.

The breast cancer that may be treated or prevented by the method may be a basal-like breast cancer such as an invasive ductal carcinoma (IDC). In some embodiments, the breast cancer is negative for oestrogen receptors (ER⁻), progesterone receptors (PR⁻), and HER2 (HER2⁻) (ie “triple negative” breast cancer (TNBC)) including, for example, triple negative invasive ductal carcinoma.

In some embodiments, the breast cancer is associated with elevated levels of either one or both of IL-3R and IL-3 in the subject. As used herein, references to elevated levels of either one or both of IL-3R and IL-3 refers to elevated levels in the subject relative to the median level of IL-3R or IL-3 in a healthy population. As such, elevated levels of IL-3R include either one or both of gene expression levels for either one or both of an IL-3R α chain or an IL-3R β_{c} chain that are greater than or equal to 1.5-fold higher than the median level in a healthy population and levels of the receptor on the surface of a target cell that are greater than or equal to 1.5-fold higher than the median level on a healthy cell. Elevated levels of IL-3 include either one or both of gene expression levels that are greater than or equal to 1.5-fold higher than the median level in a healthy population and protein levels that are greater than or equal to 1.5-fold higher than the median level in a healthy population. Subjects with an elevated IL-3R level can be identified by, for example, performing a standard assay for IL-3R (eg using an automated antibody detection system) on a suitable body sample (eg a tumour biopsy sample). Subjects with an elevated IL-3 level can be identified by, for example, performing a standard assay for IL-3 (eg an IL-3 ELISA) on a suitable body sample (eg whole blood, serum, or a tumour biopsy sample).

Breast cancer that is associated with elevated levels of either one or both of IL-3R and IL-3 may indicate that the breast cancer has vascular potential or that the breast cancer cells are VM competent. Thus, in some embodiments, the breast cancer is considered as having vascular potential or being VM competent. Accordingly, the method of the first aspect may further comprise a pre-treatment step (ie a step prior to administering an IL-3 inhibiting agent) comprising determining the breast cancer of the subject as having vascular potential or being VM competent by detecting an elevated level of either one or both of IL-3R and IL-3 as described in the preceding paragraph. This pre-treatment step may also involve detecting in a breast cancer cell-containing sample (eg a tumour biopsy sample) one or more of VE-cadherin (CD144), the MUC18 glycoprotein (CD146), platelet endothelial cell adhesion molecule (PECAM-1/CD31), Tie-2 and VEGFR2.

The method of the first aspect involves the administration of an IL-3-inhibiting agent. Such an agent preferably inhibits or abrogates the IL-3-IL-3R signalling axis (ie the agent inhibits or abrogates signalling downstream of IL-3R by, for example, inhibiting the binding of IL-3 with IL-3R to prevent receptor activation).

In some embodiments, the IL-3 inhibiting agent may inhibit the activity of endogenous IL-3 and/or IL-3R. As such, the agent may be selected from anti-IL-3 receptor (anti-IL-3R) antibodies or IL-3R-binding fragments thereof (eg Fab fragments or recombinant scFv fragments), anti-IL-3 antibodies or IL-3-binding fragments thereof (eg Fab fragments or recombinant scFv fragments), soluble extra-cytoplasmic receptor domains of IL-3 receptors (eg the N-terminal extracellular domain of the IL-3Rα chain at amino acids 19-305), other soluble molecules or matrix-associated proteins that bind to IL-3 (eg interferon-alpha³⁰), and peptide, peptide mimetic, and small organic molecule inhibitors of, for example, IL-3 binding to its receptor or, additionally or alternatively, IL-3R phosphorylation, transmission of signalling information from the IL-3R to the cell nucleus, and the activity of relevant transcription factor(s) on the cell genome.

In other embodiments, the IL-3 inhibiting agent may decrease the amount of endogenous IL-3 in the subject (particularly, the serum level of endogenous IL-3), and may be selected from agents comprising anti-IL-3 antibodies or IL-3-binding fragments thereof (eg Fab fragments or recombinant scFv fragments), catalytic and inhibitory oligonucleotide molecules targeted against the IL-3 gene (eg ribozymes, DNAzymes, antisense RNA, and small inhibitory RNA (siRNA)), and inhibitors of IL-3 transcription or translation (eg NF-IL3-A²⁹).

Preferably, the IL-3 inhibiting agent binds to the IL-3R α chain or β_{c} chain to inhibit binding of IL-3 to IL-3R. Suitable examples of such an agent may bind to site 1 of the α chain²⁸ of the IL-3R or to site 2 of the β_{c} chain²⁸ of the IL-3R. The β chain of the IL-3R is a subunit that is shared (ie “common”; thereby denoted as β_{c}) with other cytokine receptors, such as the GM-CSF and IL-5 receptors, and which uses a multi-purpose site 2 recognition cytokine homology region (CHR) that is cross-specific to each of these other cytokine receptors²⁸. Binding of the IL-3 inhibiting agent to site 2 of the β_{c} chain may thereby also inhibit or abrogate the cytokine-cytokine receptor axis for other cytokine receptors that share the β_{c} chain.

Preferably, the IL-3-inhibiting agent is an agent comprising an anti-IL-3R antibody or IL-3R-binding fragment thereof or an anti-IL-3 antibody or IL-3-binding fragment thereof. Such antibodies and fragments are considered to be inhibitory antibodies and antibody fragments (or, in other words, neutralising antibodies and antibody fragments).

More preferably, the IL-3-inhibiting agent is an agent comprising an inhibitory humanised monoclonal anti-IL-3R antibody, an inhibitory humanised monoclonal anti-IL-3 antibody, an inhibitory fully human monoclonal anti-IL-3R antibody or an inhibitory fully human monoclonal anti-IL-3 antibody. Humanised anti-IL-3R and anti-IL-3 antibodies may be produced in accordance with any of the methods well known to those skilled in the art including, for example, the methodology described in U.S. Pat. No. 5,225,539 (the entire disclosure of which is incorporated herein by reference), by specificity determining residue (SDR) grafting as described in Kashmiri, Syed V. S. et al. “SDR grafting—a new approach to antibody humanization”, Methods, 36(1): 25-34 (2005) (the entire disclosure of which is incorporated herein by reference), by affinity maturation using phage display as described in Marvin, Jonathan S. and Henry B. Lowman. “Antibody humanization and affinity maturation using phage display”, Phage Display in Biotechnology and Drug Discovery (2015) (the entire disclosure of which is incorporated herein by reference), using heavy chain complementarity-determining region 3 grafting coupled with in vitro somatic hypermutation as described in Bowers, Peter M. et al. “Humanization of antibodies using heavy chain complementarity-determining region 3 grafting coupled with in vitro somatic hypermutation”, Journal of Biological Chemistry 288(11):7688-7696 (2013) (the entire disclosure of which is incorporated herein by reference) or any other suitable method for producing humanised antibodies. Fully human anti-IL-3R and anti-IL-3 antibodies may be produced in accordance with any of the methods well known to those skilled in the art including, for example, using transgenic mice or phage display as described in Lonberg, N. “Fully human antibodies from transgenic mouse and phage display platforms”, Current Opinion in Immunology 20:450-459 (2008).

In some embodiments, the IL-3 inhibiting agent is an agent comprising either one or both of the anti-IL-3Rα antibody, 7G3 (targeted against site 1 in the IL-3R α chain) and the anti-IL-3Rβ antibody, BION-1 (targeted against the membrane proximal domain).

In some embodiments, it may be desirable to modify the IL-3 inhibiting agent to increase its serum half-life. It may be particularly desirable to modify the IL-3 inhibiting agent to increase its serum half-life, where the IL-3 inhibiting agent is an antibody or antibody fragment. Prolonging the half-life of the IL-3 inhibiting agent may reduce the amount and/or frequency of dosing, increase plasma residence time, decrease clearance and increase clinical activity in vivo. Accordingly, the IL-3-inhibiting agent may further comprise polyalkane glycol (eg polyethylene glycol (PEG), and/or polypropylene glycol (PPG)), carbohydrate polymer, amino acid polymer, polyvinyl pyrrolidone, recombinant PEG mimetic, colominic acid, hydroxyethyl starch, carbohydrate (ie via glycosylation), serum albumin or at least a serum albumin binding domain or peptide, transferrin, transferrin receptor or at least the transferrin-binding portion thereof, or any other molecule operable to increase the half-life of the IL-3 inhibiting agent. Antibodies with improved in vivo half-lives and methods for preparing them are disclosed in, for example, U.S. Pat. No. 6,277,375, International Publication No. WO 98/23289 and Kontermann, R. “Strategies to Extend Plasma Half-Lives of Recombinant Antibodies”, BioDrugs 23(2):93-109 (2009) (the entire disclosure of these documents is to be regarded as incorporated herein by reference).

While not wishing to be bound by theory, it is considered that the method of the first aspect is useful for treating or preventing breast cancer in a subject by inhibiting or preventing vasculogenesis and/or vasculogenic mimicry (VM) in a tumour associated with breast cancer. The method may thereby attenuate breast cancer growth and/or progression of the breast cancer to a more advanced stage. It is also considered that the method may inhibit tumour metastasis.

Preferably, the method of the first aspect is used for the treatment of a subject suffering from triple negative invasive ductal carcinoma, where a tumour mass involved in vasculogenesis and/or vasculogenic mimicry leads to an elevated level of either one or both of IL-3R and IL-3.

The method of the first aspect may further comprise administering one or more additional agent(s) for the treatment of cancer. For example, the IL-3-inhibiting agent may be used in combination with other anti-cancer agents (eg Bevacizumab or other anti-angiogenic agents) or agents intended to make cancer cells more susceptible to anti-cancer therapies (eg chemotherapy and radiotherapy). Where used in combination with other anti-cancer agents, the IL-3-inhibiting agent and the other anti-cancer agent can be administered in the same pharmaceutical composition or in separate pharmaceutical compositions. If administered in separate pharmaceutical compositions, the IL-3-inhibiting agent and the other anti-cancer agent may be administered simultaneously or sequentially in any order (eg within seconds or minutes or even hours (eg 2 to 48 hours)).

The method of the first aspect will typically be applied to the treatment of breast cancer in a human subject. However, the subject may also be selected from, for example, livestock animals (eg cows, horses, pigs, sheep and goats), companion animals (eg dogs and cats) and exotic animals (eg non-human primates, tigers, elephants etc).

In a second aspect, the present disclosure provides a method of diagnosing or prognosing breast cancer in a subject, said method comprising detecting an elevated level of either one or both of IL-3R and IL-3 present in a suitable body sample of said subject.

The detection of an elevated level of either one or both of IL-3R and IL-3 in accordance with the method of the second aspect may provide information of diagnostic and/or prognostic value such as, for example, information regarding a characteristic of the breast cancer (eg the breast cancer type, level of aggressiveness and/or likelihood of progression to a more advanced stage including metastasis) and/or a risk that the breast cancer is invasive ductal carcinoma, in which case, a prognosis of a poor clinical outcome may be made absent successful medical intervention. In some embodiments, the detection of an elevated level of either one or both of IL-3R and IL-3 may indicate that the breast cancer has vascular potential or that the breast cancer cells are VM competent. In some embodiments, the detection of an elevated level of either one or both of IL-3R and IL-3 may indicate a risk that the breast cancer is a triple negative cancer such as a triple negative invasive ductal carcinoma. In some embodiments, elevated levels of IL-3R include either one or both of gene expression levels for either one or both of an IL-3 α chain or an IL-3R β chain that are greater than or equal to 1.5-fold higher than the median level in a healthy population (ie the median level in a representative sample of persons from a population that do not suffer from breast cancer) and levels of the receptor on the surface of a target cell that are greater than or equal to 1.5-fold higher than the median level on healthy cells (ie the median level on cells of the same type or from the same tissue type which have been isolated from persons that do not suffer from breast cancer). In some embodiments, elevated levels of IL-3 include either one or both of gene expression levels that are greater than or equal to 1.5-fold higher than the median level in a healthy population and protein levels that are greater than or equal to 1.5-fold higher than the median level in a healthy population.

In some embodiments, the method comprises obtaining a suitable body sample (eg whole blood, serum or a tumour biopsy sample) from the subject, providing an IL-3R binding agent (eg an anti-IL-3R antibody), contacting the sample under conditions to form a complex comprising IL-3R and the IL-3R binding agent (that is, if IL-3R is present), and detecting the complex. As such, the method may comprise using flow cytometry with an antibody for IL-3R. In some embodiments, the method comprises obtaining a suitable body sample (eg whole blood, serum or a tumour biopsy) from the subject, providing an IL-3 binding agent (eg an anti-IL-3 antibody), contacting the sample under conditions to form a complex comprising IL-3 and the IL-3 binding agent (that is, if IL-3 is present), and detecting the complex. As such, the method may comprise using an ELISA for IL-3. Preferably, the binding agent binds specifically to either one of IL-3R or IL-3. As used herein, the term “binds specifically” or “specific binding” means that the binding agent should not bind substantially to (that is, substantially “cross-react” with) another peptide, polypeptide or substance present in the suitable body sample. Preferably, the specifically bound IL-3 or IL-3R will be bound with at least 3 times higher, more preferably at least 10 times higher, and most preferably at least 50 times higher affinity than any other relevant peptide, polypeptide or substance. Non-specific binding may be tolerable, if it can still be distinguished and measured unequivocally, for example, according to its size on a Western Blot, or by the relatively higher abundance of IL-3R or IL-3 in the sample, or if it can be controlled for using a negative control sample or a normal subject(s) control sample.

A variety of assays may be suitable for determining the amount of either one or both of IL-3R and IL-3 in a suitable body sample. In an in vitro method, the amount of IL-3R present in a suitable body sample may be readily determined by any suitable method including, for example, immunoassays such as enzyme-linked immunosorbant assay (ELISA), radioimmunoassay (RIA) and immunohistochemistry (eg with sectionalised samples of a tissue biopsy and by fixing the cells without detergent such that the plasma membrane remains intact) using anti-IL-3R antibodies or fragments thereof. Similarly, the amount of IL-3 present in a suitable body sample may be readily determined by any suitable method including, for example, immunoassays such as ELISA, RIA and immunohistochemistry (eg with sectionalised samples of a tissue biopsy) using anti-IL-3 antibodies or fragments thereof. Particularly suitable methods for determining the amount of either one or both of IL-3R and IL-3 present in a suitable body sample are immunoassays utilising labelled molecules in various sandwich, competition, or other assay formats. Such immunoassays will develop a signal which is indicative for the presence or absence of either one or both of IL-3R and IL-3. Further, the strength of the signal generated by such immunoassays may be correlated directly or indirectly (for example, reversely proportional) to the amount of either one or both of IL-3R and IL-3 present in a sample. Other particularly suitable methods for determining the amount of IL-3 present in a suitable body sample are methods comprising the measurement of a physical or chemical property specific for IL-3 such as a precise molecular mass or nuclear magnetic resonance (NMR) spectrum. Such methods may, therefore, be conducted using biosensors, optical devices coupled to immunoassays, biochips, analytical devices such as mass-spectrometers, NMR-analysers and chromatography devices. Further particularly suitable methods for determining the amount of IL-3 present in a suitable body sample include microplate ELISA-based methods, fully-automated or robotic immunoassays (available, for example, on Elecsys® analysers; Roche Diagnostics Corporation, Indianapolis, Ind., United States of America), enzymatic Cobalt Binding Assay (CBA) (available, for example, on Roche-Hitachi analysers; Roche Diagnostics Corporation) and latex agglutination assays (available, for example, on Roche-Hitachi analysers). Still further examples of particularly suitable methods for determining the amount of IL-3 present in a suitable body sample include methods involving precipitation (eg immunoprecipitation), electrochemiluminescence (ie electro-generated chemiluminescence), electrochemiluminescence sandwich immunoassays (ECLIA), dissociation-enhanced lanthanide fluoro immuno assay (DELFIA), scintillation proximity assay (SPA), turbidimetry, nephelometry, latex-enhanced turbidimetry and nephelometry. Further methods that are well known to persons skilled in the art, such as gel electrophoresis, Western Blotting and mass spectrometry, may also be used alone or in combination with other suitable methods as described above.

As such, the determination of the amount of either one or both of IL-3R and IL-3 in the suitable body sample may comprise the steps of (i) contacting either one or both of IL-3R and IL-3 with a specific binding agent, (ii) optionally removing non-bound binding agent, and (iii) measuring the amount of bound binding agent. The bound binding agent (which may be bound by covalent and/or non-covalent binding) will generate an intensity signal. As indicated above, the binding agent may be selected from either one or both of anti-IL-3R and anti-IL-3 antibodies or fragments thereof but might otherwise be selected from any other binding agents that may bind either one or both of IL-3R and IL-3 such as, for example, any compound (including peptides, polypeptides, nucleic acids, aptamers (eg nucleic acid or peptide aptamers), and small molecules) that bind to either one or both of IL-3R and IL-3. However, preferably, the binding agent is selected from either one or both of anti-IL-3R and anti-IL-3 antibodies or fragments thereof (including polyclonal and monoclonal antibodies, as well as fragments thereof, such as Fv, Fab and F(ab)₂ fragments that are capable of binding either one or both of anti-IL-3R and anti-IL-3, and recombinant antibodies such as single chain antibodies (eg scFV antibodies)). Methods of preparing such binding agents are well known to those skilled in the art.

The binding agent may be coupled covalently or non-covalently to a label allowing detection and measurement of the binding agent. Suitable labelling may be performed by any of the direct or indirect methods well known to those skilled in the art. However, by way of brief explanation, direct labelling involves the coupling of the label directly (ie covalently or non-covalently) to the binding agent, while indirect labelling involves the binding (ie covalently or non-covalently) of a secondary binding agent to the binding agent (ie “primary binding agent”) wherein the secondary binding agent should specifically bind to the first binding agent and may be coupled with a suitable label and/or be the target (receptor) of tertiary binding agent binding to the secondary binding agent. The use of secondary, tertiary or even higher order binding agents can be used to increase the signal. Suitable secondary and higher order binding agents may include antibodies, secondary antibodies, and the well-known streptavidin-biotin system (Vector Laboratories, Inc, Burlingame, Calif., United States of America). The binding agent may also be “tagged” with one or more tags well known to those skilled in the art, which tags may then be targets for higher order binding agents. Suitable tags include biotin, digoxygenin, His-Tag, glutathione-S-transferase, FLAG, Green Fluorescent Protein (GFP), myc-tag, Influenza A virus haemagglutinin (HA), maltose binding protein and the like. Where the binding agent is a protein, peptide or polypeptide, the tag is preferably located at the N-terminus and/or C-terminus. Suitable labels include any labels that are detectable by an appropriate detection method such as, for example, gold particles, latex beads, acridan ester, luminol, ruthenium, enzymatically-active labels, radioactive labels, magnetic labels (for example, “magnetic beads”, including paramagnetic and superparamagnetic labels), and fluorescent labels. Suitable enzymatically-active labels include, for example, horseradish peroxidase, alkaline phosphatase, β-galactosidase, luciferase and derivatives thereof. Suitable substrates for enzymatically-active labels to enable detection include di-amino-benzidine (DAB), 3,3′-5,5′-tetramethylbenzidine, 4-nitro blue tetrazolium chloride and 5-bromo-4-chloro-3-indolyl-phosphate (NBT-BCIP), available as a ready-made stock solution from Roche Diagnostics Corporation), CDP-Star™ (Amersham Biosciences Inc, Fairfield, Conn., United States of America), and ECF™ (Amersham Biosciences Inc). Suitable radioactive labels include ³⁵S, ¹²⁵I, ³²P, ³³P and the like. Radioactive labels can be detected by any of the methods well known to those skilled in the art including, for example, a light-sensitive film or a phosphor imager. Suitable fluorescent labels include fluorescent proteins (such as GFP and derivatives thereof, Cy3, Cy5, Texas Red, Fluorescein and the Alexa dyes (eg Alexa 568)). The use of quantum dots as fluorescent labels is also contemplated.

In some embodiments, the amount of either one or both of anti-IL-3R and anti-IL-3 in a suitable body sample may be determined as follows:

(i) contacting a solid support comprising a binding agent for either one or both of anti-IL-3R and anti-IL-3 as described above with said suitable body sample comprising either one or both of anti-IL-3R and anti-IL-3 and thereafter (ii) measuring the amount of either one or both of anti-IL-3R and anti-IL-3 which has become bound to the support. Preferably, in such embodiments, the binding agent is selected from the group of binding agents consisting of nucleic acids, peptides, polypeptides, antibodies and aptamers, and, preferably, is provided on the solid support in an immobilised form. The solid support may be composed of any of the typical materials well known to those skilled in the art including, inter alia, commercially available column materials, polystyrene beads, latex beads, magnetic beads, colloid metal particles, glass and/or silicon chips and surfaces, nitrocellulose strips, membranes, sheets, duracytes, wells and walls of suitable reaction trays such as 96-well plates and other plates, plastic tubes etc. The binding agent used in such embodiments may also be bound to a suitable carrier such as glass, polystyrene, polyvinyl chloride (PVC), polypropylene, polyethylene, polycarbonate, dextran, nylon, amyloses, natural and modified celluloses, polyacrylamides, agaroses and magnetite. The nature of the carrier can be either soluble or insoluble. Suitable methods for immobilising the binding agent to the solid support are well known to those skilled in the art and include, for example, ionic, hydrophobic, covalent interactions and the like. It is also contemplated to use “suspension arrays”³¹, wherein a carrier such as a microbead or microsphere is present in suspension and the array consists of different microbeads or microspheres, possibly labelled, carrying different binding agents. Methods of producing such arrays, for example based on solid-phase chemistry and photo-labile protective groups, are well known to those skilled in the art (see, for example, U.S. Pat. No. 5,744,305).

In some embodiments of the method of the second aspect, the method may further comprise detecting one or more of VE-cadherin (CD144), the MUC18 glycoprotein (CD146), platelet endothelial cell adhesion molecule (PECAM-1/CD31), Tie-2 and VEGFR2.

In a third aspect, the present disclosure provides the use of an IL-3-inhibiting agent for the manufacture of a medicament for the therapeutic treatment of breast cancer.

The medicament may be suitable for treating or preventing a breast cancer such as, for example, a basal-like breast cancer such as an invasive ductal carcinoma (IDC). In some embodiments, the breast cancer is a triple negative breast cancer such as a triple negative invasive ductal carcinoma. In some embodiments, the breast cancer is considered as having vascular potential or being VM competent. The IL-3 inhibiting agent is an IL-3 inhibiting agent as described above. Preferably, the IL-3-inhibiting agent is an inhibitory humanised monoclonal anti-IL-3R antibody, an inhibitory humanised monoclonal anti-IL-3 antibody, an inhibitory fully human monoclonal anti-IL-3R antibody or an inhibitory fully human monoclonal anti-IL-3 antibody. As indicated above, fully human monoclonal antibodies may be prepared using, for example, transgenic mice or phage display as described in Lonberg, N. “Fully human antibodies from transgenic mouse and phage display platforms”, Current Opinion in Immunology 20:450-459 (2008).

In a fourth aspect, the present disclosure provides the use of an IL-3-inhibiting agent for the treatment of breast cancer.

The use may be suitable for treating or preventing a breast cancer such as, for example, a basal-like breast cancer such as an invasive ductal carcinoma (IDC). In some embodiments, the breast cancer is a triple negative breast cancer such as a triple negative invasive ductal carcinoma. In some embodiments, the breast cancer is considered as having vascular potential or being VM competent. The IL-3 inhibiting agent is an IL-3 inhibiting agent as described above. Preferably, the IL-3-inhibiting agent is an inhibitory humanised monoclonal anti-IL-3R antibody, an inhibitory humanised monoclonal anti-IL-3 antibody, an inhibitory fully human monoclonal anti-IL-3R antibody or an inhibitory fully human monoclonal anti-IL-3 antibody.

In a fifth aspect, the present disclosure relates to the use of an inhibitory humanised monoclonal anti-IL-3R antibody, an inhibitory humanised monoclonal anti-IL-3 antibody, an inhibitory fully human monoclonal anti-IL-3R antibody or an inhibitory fully human monoclonal anti-IL-3 antibody for the treatment of invasive ductal carcinoma (including invasive ductal carcinoma having vascular potential or being VM competent).

In a sixth aspect, the present disclosure provides a method for the prevention or treatment of metastasis in a subject suffering from breast cancer, said method comprising administering to said subject an IL-3-inhibiting agent.

In some embodiments, the breast cancer is metastatic and has spread to areas of the body outside the breast, such as the bone. In some embodiments, the breast cancer is a triple negative breast cancer such as a triple negative invasive ductal carcinoma. In some embodiments, the breast cancer is considered as having vascular potential or being VM competent. The IL-3 inhibiting agent is an IL-3 inhibiting agent as described above. Preferably, the IL-3-inhibiting agent is an inhibitory humanised monoclonal anti-IL-3R antibody, an inhibitory humanised monoclonal anti-IL-3 antibody, an inhibitory fully human monoclonal anti-IL-3R antibody or an inhibitory fully human monoclonal anti-IL-3 antibody.

In a seventh aspect, the present disclosure provides a method for the stratification of breast cancer, said method comprising detecting an elevated level of either one or both of IL-3R or IL-3 present in a suitable body sample of said subject.

The detection of an elevated level of either one or both of IL-3R and IL-3 in accordance with the method of the seventh aspect may provide information of diagnostic and/or prognostic value such as, for example, information regarding a characteristic of the breast cancer (eg the breast cancer type, level of aggressiveness and/or likelihood of progression to a more advanced stage including metastasis) and/or a risk that the breast cancer is invasive ductal carcinoma, in which case, a prognosis of a poor clinical outcome may be made absent successful medical intervention, and/or information to further stratify breast cancers beyond different molecular subtypes (eg luminal A, luminal B, and basal-like) such as whether or not the breast cancer has vascular potential or that the breast cancer cells are VM competent. Based on the stratifications, breast cancer patients may receive targeted therapy. The method of the seventh aspect may be performed substantially in accordance with the steps of the method of the second aspect. In some embodiments of the method of the seventh aspect, the method may further comprise detecting one or more of VE-cadherin (CD144), the MUC18 glycoprotein (CD146), platelet endothelial cell adhesion molecule (PECAM-1/CD31), Tie-2 and VEGFR2.

IL-3-inhibiting agents for use in the method or uses of the present disclosure may be formulated into any suitable pharmaceutical/veterinary composition or dosage form (eg medicaments for oral, buccal, nasal, intramuscular and intravenous administration). Typically, such a composition will be administered to the subject in an amount which is effective to achieve any one or more of attenuating breast cancer, decreasing the amount of endogenous IL-3 and inhibiting the activity of endogenous IL-3, and may, for example, comprise a therapeutically effective amount of the IL-3-inhibiting agent. It will be understood by those skilled in the art that the therapeutically effective amount of the IL-3-inhibiting agent may vary and depend upon a variety of factors including the activity of the particular agent, the metabolic stability and length of action of the particular agent, the age, body weight, sex and/or health of the subject, the route and time of administration, rate of excretion of the particular agent, and the severity of the cancer to be treated. A suitable composition may be intended for single daily administration, multiple daily administration, or controlled or sustained release, as needed to achieve the most effective results.

Pharmaceutical compositions comprising the IL-3 inhibiting agent may also contain physiologically acceptable carriers, excipients or stabilisers (Remington's Pharmaceutical Sciences 16th edition, Osul, A. Ed. (1980)). Acceptable carriers, excipients, or stabilisers are nontoxic to a subject at the dosages and concentrations employed, and may include buffers such as phosphate, citrate, histidine and other organic acids; antioxidants including ascorbic acid and methionine; preservatives (such as octadecyldimethylbenzyl ammonium chloride; hexamethonium chloride; benzalkonium chloride, benzethonium chloride); phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (eg Zn-protein complexes); and/or non-ionic surfactants such as TWEEN™, PLURONICS™ or PEG.

The finding that breast cancer tumours undergoing vasculogenic mimicry overexpress IL-3 suggests that methods of gene therapy to decrease the level of IL-3 in a subject may provide an effective treatment of breast cancer. Therefore, the present disclosure also contemplates gene therapy methods, and gene therapy agents, for preventing or inhibiting vasculogenesis and/or vasculogenic mimicry and thereby attenuating breast cancer growth, comprising recombinant IL-3 suppressive genes to bring about decreased endogenous IL-3 expression. Vectors suitable for the introduction of IL-3 suppressive genes include recombinant adenoviral or adenoviral-associated vectors, recombinant retroviral vectors, recombinant lentivirus vectors, liposomes including linear DNA, and transduced or transformed stem cells.

Further, the present disclosure extends to kits for use in a method or use according to any one or more of the above aspects. Such a kit may comprise one or more packaged therapeutic agent (eg an IL-3-inhibiting agent such as an anti-IL-3 antibody) and/or diagnostic or prognostic agent (eg an agent for the detection of an elevated level of IL-3R or IL-3 such as an anti-IL-3R binding agent or an anti-IL-3 binding agent (such as described above). The kit may include instructions for use of the therapeutic agent and/or diagnostic or prognostic agent in a method or use according to any one or more of the above aspects.

Still further, the present disclosure extends to the use of anti-IL-3R binding agent or an anti-IL-3 binding agent (such as described above) in the manufacture of a diagnostic or prognostic agent for diagnosing or prognosing breast cancer in a subject (eg by detecting an elevated level of either one or both of IL-3R and IL-3 present in a suitable body sample of a subject) or stratification of breast cancer (eg by detecting an elevated level of either one or both of IL-3R or IL-3 present in a suitable body sample of a subject).

In an eighth aspect, the present disclosure extends to a method of treating or preventing a cancer associated with elevated levels of either one or both of IL-3R and IL-3 in a subject, said method comprising administering to said subject an interleukin-3 (IL-3)-inhibiting agent, such as, for example, an anti-IL-3R antibody. Apart from breast cancer as described above, other cancers that may be associated with elevated levels of either one or both of IL-3R and IL-3 may include, for example, solid tumour cancers such as renal cell carcinoma,³³ brain cancer^{34, 35} and lung carcinoma,^{36, 37} patients of which have been observed with elevated circulating levels of IL-3.³⁸ Those skilled in the art will recognise that a cancer-suffering subject with an elevated IL-3R and/or IL-3 level can be identified by, for example, performing a standard assay for IL-3R (eg using an automated antibody detection system) on a suitable body sample (eg a tumour biopsy sample) as described above. Elevated levels of IL-3R include either one or both of gene expression levels for either one or both of an IL-3R α chain or an IL-3R β_{c} chain that are greater than or equal to 1.5-fold higher than the median level in a healthy population and levels of the receptor on the surface of a target cell that are greater than or equal to 1.5-fold higher than the median level on a healthy cell. Elevated levels of IL-3 include either one or both of gene expression levels that are greater than or equal to 1.5-fold higher than the median level in a healthy population and protein levels that are greater than or equal to 1.5-fold higher than the median level in a healthy population. A cancer-suffering subject showing elevated levels of either one or both of IL-3R and IL-3 may have a cancer (eg a solid tumour cancer) that has vascular potential or cancer cells that are VM competent. As would be appreciated by those skilled in the art, the method of the eighth aspect may be performed in accordance with the above described first aspect, having regard to variations appropriate to the particular cancer type.

In a ninth aspect, the present disclosure provides a method of diagnosing or prognosing cancer associated with elevated levels of either one or both of IL-3R and IL-3 in a subject, said method comprising detecting an elevated level of IL-3R or IL-3-present in a suitable body sample of said subject. As would be appreciated by those skilled in the art, the method of the ninth aspect may be performed in accordance with the above described second aspect, having regard to variations appropriate to the particular cancer type.

In a tenth aspect, the present disclosure provides the use of an IL-3-inhibiting agent for the manufacture of a medicament for the therapeutic treatment of cancer associated with elevated levels of either one or both of IL-3R and IL-3. As would be appreciated by those skilled in the art, the method of the tenth aspect may be performed in accordance with the above described third aspect, having regard to variations appropriate to the particular cancer type.

In an eleventh aspect, the present disclosure provides the use of an IL-3-inhibiting agent for the treatment of cancer associated with elevated levels of either one or both of IL-3R and IL-3. As would be appreciated by those skilled in the art, the method of the eleventh aspect may be performed in accordance with the above described fourth aspect, having regard to variations appropriate to the particular cancer type.

In a twelfth aspect, the present disclosure relates to the use of an inhibitory humanised monoclonal anti-IL-3R antibody, an inhibitory humanised monoclonal anti-IL-3 antibody, an inhibitory fully human monoclonal anti-IL-3R antibody or an inhibitory fully human monoclonal anti-IL-3 antibody for the treatment of renal cell carcinoma, brain cancer and lung carcinoma. As would be appreciated by those skilled in the art, the method of the twelfth aspect may be performed in accordance with the above described fifth aspect, having regard to variations appropriate to the particular cancer type.

In a thirteenth aspect, the present disclosure provides a method for the prevention or treatment of metastasis in a subject suffering from renal cell carcinoma, brain cancer or lung carcinoma, said method comprising administering to said subject an IL-3-inhibiting agent. As would be appreciated by those skilled in the art, the method of the thirteenth aspect may be performed in accordance with the above described sixth aspect, having regard to variations appropriate to the particular cancer type.

In a fourteenth aspect, the present disclosure provides a method for the stratification of renal cell carcinoma, brain cancer or lung carcinoma, said method comprising detecting an elevated level of IL-3R or IL-3 present in a suitable body sample of said subject. The method of the fourteenth aspect may provide information to further stratify cancers beyond different molecular subtypes such as whether or not the cancer has vascular potential or that the cancer cells are VM competent. As would be appreciated by those skilled in the art, the method of the fourteenth aspect may be performed in accordance with the above described seventh aspect, having regard to variations appropriate to the particular cancer type.

In order that the nature of the present disclosure may be more clearly understood, preferred forms thereof will now be described with reference to the following non-limiting example(s).

### EXAMPLES

### Example 1 Targeting the IL-3 Receptor to Prevent or Treat Breast Cancer

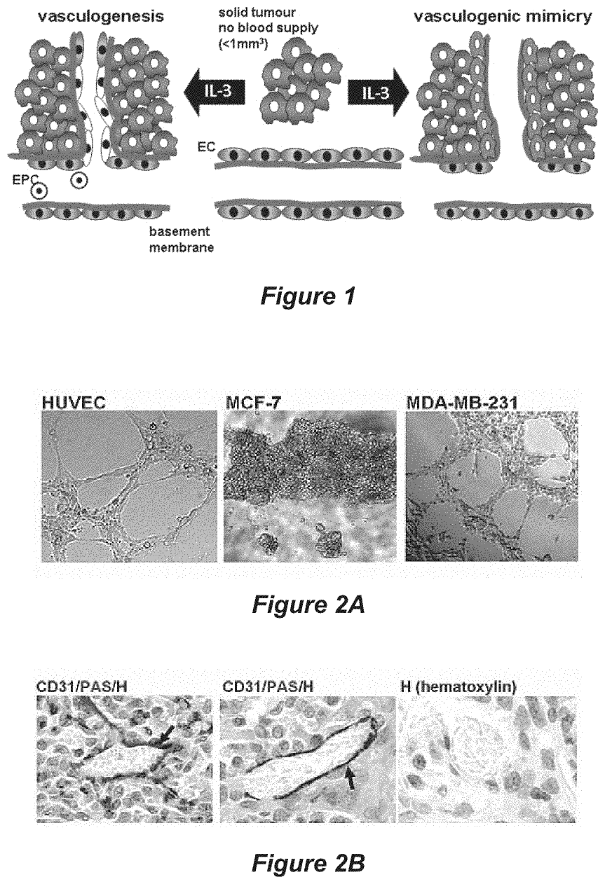

A study was conducted with two aims: 1) to determine the role of IL-3 in vasculogenic mimicry by breast cancer cells; and 2) to evaluate the impact of blocking the IL-3/IL-3R signalling axis in primary human breast cancers to attenuate IDC progression in vivo. A model for the role of IL-3 in vasculogenesis and vasculogenic mimicry is shown in FIG. 1.

Vasculogenesis in Cancer

In response to tumour growth, endothelial progenitor cells (EPCs) migrate from the bone marrow to the periphery, where they proliferate and differentiate into mature endothelial cells (ECs) for expansion of the local vasculature. Current protocols for EPC identification employ combinations of progenitor cell markers (CD133 and CD34) and the endothelial cell markers (VEGFR2 and CD31)¹¹. The present inventors recently identified a distinct population of circulating, non-adherent CD133⁺CD34⁺VEGFR2⁺ CD31⁺ EPCs (naEPCs)¹². These human EPCs were obtained from the CD133⁺ sorted fraction of umbilical cord blood (UCB) mononuclear cells and cultured for 4 days in a defined media. Functional studies confirmed the EPC phenotype with cells (i) binding Ulex europaeus lectin (UEA-1), (ii) taking up acetylated-low density lipoprotein (Ac-LDL), (iii) enhancing tube formation in a 3-dimensional in vitro assay when seeded with human umbilical vein endothelial cells (HUVEC) on Matrigel (an extracellular matrix derived from murine sarcoma cells that supports vascular tube formation in vitro and thus mimics in vivo vasculogenesis)¹², and (iv) incorporating into the NOD/SCID mouse vasculature¹²; all key features of EPCs. The identification of these cells revealed critical information about EPCs, namely the expression of previously unknown or under-appreciated surface expressed proteins including the receptor for IL-3.

Vasculogenic Mimicry (VM) in Cancer

Although the mechanisms which underpin VM are yet to be fully defined, several studies have shown that VM channels can anastomose (fuse) with conventional blood vessels to access the blood supply, and possess a lumen through which blood can flow throughout the tumour¹³. It was observed that human IDC cell lines can be stratified into those that are VM competent and those that are not. An in vitro Matrigel tube forming assay was conducted using IDC cell lines HUVEC, MDA MB-231, HS-578-T, BT549, MCF7, ZR751 and SUM159 par. Approximately 1×10⁴ cells were seeded into 12 μl Matrigel and images were captured after 3-6 h. The in vitro Matrigel assay identified IDC cell lines which, like HUVEC, can form tube-like structures (ie VM). A subset of the results are shown in FIG. 2A (one of n=5) which compares HUVEC, MCF-7 and MDA-MB-231 cells. The results showed that HUVEC, MDA MB-231, HS-578-T, BT549 cells and SUM159 are VM competent and that MCF7 and ZR751 cells are not VM competent (See Table 1, below). The BT549, HS-578-T, MDA MB-231 and SUM159 cell lines are all examples of TNBC cell lines.

TABLE 1
In vitro Matrigel tube formation assay to test
VM capability of breast cancer cell lines

| | Tumour type/ | Invasive | | | | VM capable |
|---|---|---|---|---|---|---|
| Cell line | Gene cluster | nature | ER | PR | Her2 | in vitro |
| BT 549 | IDC/Basal B | High | − | − | − | Yes |
| HS-578-T | IDC/Basal B | High | − | − | − | Yes |
| MDA-MB- | Adeno C/Basal B | High | − | − | − | Yes |
| 231 | | | | | | |
| SUM159 | Anaplastic C/ | High | − | − | − | Yes |
| | Basal B | | | | | |
| MCF7 | IDC/Luminal | Low | + | + | +/− | No |
| T-47-D | IDC/Luminal | Low | + | + | − | No (n = 1) |
| ZR-75-1 | IDC/Luminal | Low | + | −/+ | + | No |

An in vivo assay was also conducted where MDA-MB-231-long metastasis 2 (LM2) cells were injected into the mammary fat pad of NOD/SCID mice. Tumours were harvested after 28 days, sectioned and subjected to Periodic Acid-Schiff (PAS) staining and immunostained for CD31. Haematoxylin (H) was used to counterstain. The results can be seen in FIG. 2B which shows one of n=5. MDA-MB-231-LM2 tumours in NOD/SCID mice contained VM (CD31⁻PAS⁺) and EC-lined channels (CD31⁺PAS⁻). The in vivo assay showed that these MDA-MB-231-LM2 tumours contained both VM channels (identified by Periodic Acid-Schiff (PAS) staining (left panel, arrow) as well as EC-lined vessels (CD31 staining (middle panel, arrow)) (FIG. 2B).

Interleukin-3: A Regulator of Vascular Development in IDC

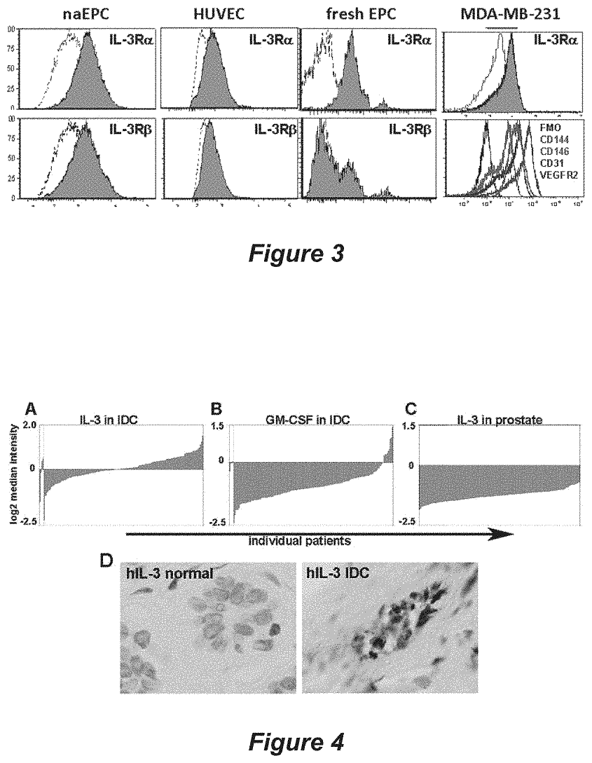

IL-3 is a pleiotropic cytokine that acts as a growth factor for several leukocyte lineages⁸. It signals through a specific IL-3 receptor that consists of two chains, an α chain which directly binds IL-3 and is specific for this growth factor, and a common β chain (β_{c}), which is shared between the receptors for IL-3, GM-CSF and IL-5 and is the major signalling component⁸. In the context of vascular biology, the present inventors have previously shown that the expression of the IL-3Rs on HUVEC is selective in that receptors for the related molecules GM-CSF and IL-5 are not detected (FIG. 3), and the IL-3R signals by stimulating EC functions^{9, 14}.

With the function of IL-3 on EPCs yet to be fully elucidated, the present inventors and others have found that IL-3 enhances naEPC proliferation (unpublished) as well as other EPCs, supports their survival^{15, 16}, and promotes EC migration and tube formation in vitro¹⁰. In order to determine whether IDCs express the IL-3Rα and β_{c} chain, fluorescence activated cell sorting (FACS) was used to analyse surface expression of the IL-3R subunits (α and β_{c}) on naEPCs, HUVEC, freshly isolated EPCs and MDA-MB-231 cells. The results are shown in FIG. 3 which shows one of n=3. Unexpectedly, the results show that IDCs express the IL-3Rα and β_{c} chain with ¹²⁵I-IL-3 binding assays detecting ˜500 receptors per cell. This is unexpected because it was not previously believed that cancer cells would be affected by IL-3 for vascular development. Both IL-3R subunits (α and β_{c}) are expressed by naEPCs as well as freshly isolated CD133⁺CD34⁺VEGFR2⁺ EPCs. The present inventors also extended previous reports of EC markers on these cells¹³ with detection of VE-cadherin (CD144), the MUC18 glycoprotein (CD146), platelet endothelial cell adhesion molecule (PECAM-1/CD31) and VEGFR2 (FIG. 3).

The finding that MDA-MB-231 are VM competent, together with data showing that MDA-MB-231 cells express the EC markers CD144, CD146, CD31, Tie-2 and VEGFR2, supports the notion that common processes exist between VM and EPCs/ECs which underpin their pro-vascular nature and that IL-3 is a major unifying factor that regulates these processes.

Interleukin-3: Selective Expression in IDC Patients

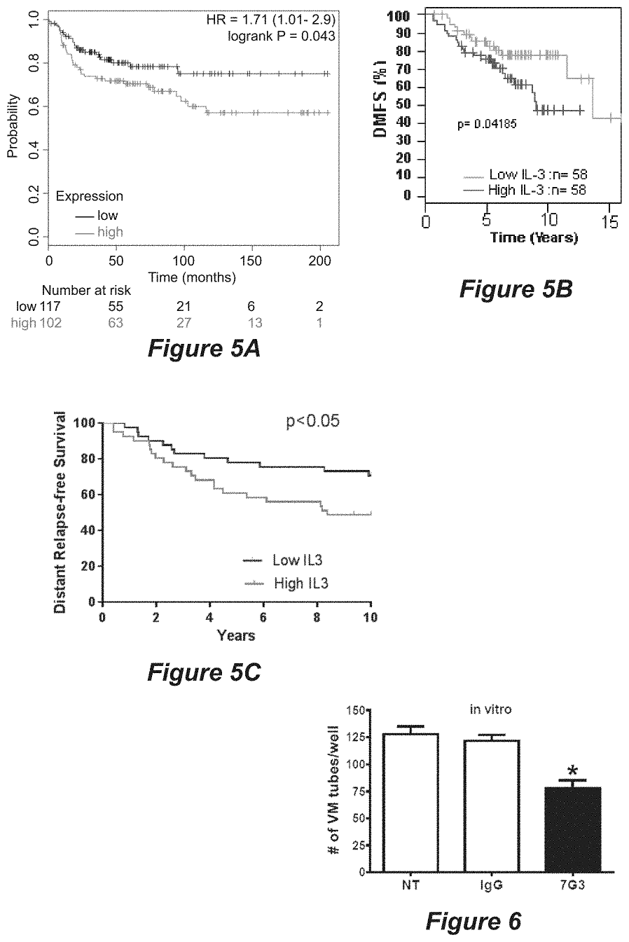

Based on this information, the present inventors hypothesised that if the IL-3/IL-3R system was involved in VM in breast cancer, then there would have to be a source of IL-3 in these patients. To investigate this, an in silico comparison of IL-3 and granulocyte-macrophage colony-stimulating factor (GM-CSF) mRNA transcript levels in tumour and normal breast tissues was conducted using datasets from the Oncomine database (Compendia Biosciences; Ann Arbor, Mich., United States of America). Of the 27 major cancer types (90 datasets) analysed against normal tissue, a significant upregulation of IL-3 was only repeatedly observed in breast cancer (7 datasets), more specifically in IDC patients where ˜50% exhibited an increase in IL-3 mRNA from laser-captured cancerous tissue^{17, 21} (FIG. 4A). This same cohort of patients did not show an increase in transcripts of the closely related GM-CSF (FIG. 4B). Notably, IL-3 gene expression in other cancer groups (eg prostate cancer) was not significantly elevated (FIG. 4C), suggesting that this increased expression of IL-3 in IDC is disease-specific. IDC tumour sections were then immunostained for IL-3 protein. FIG. 4D shows that IL-3 appeared clustered in IDC tumour sections, but not in normal breast tissue. Identification of IL-3, as well as IL-3 receptors (α and β_{c}), was also detected in the MDA-MB-231 tumours resected from mice. Using an in silico analysis of IL-3 gene expression in breast cancer patients using the publically available Kaplan-Meier Plotter program, three plots of basal breast cancer patients with high versus low IL-3 gene expression were generated, examining overall survival (FIG. 5A), distant relapse-free survival (FIG. 5B) and distant metastasis-free survival (FIG. 5C). The data for the analyses were obtained from Gene Expression Omnibus (GEO) Datasets from Gyorffy et al.²⁵ (FIG. 5A), GSE22220²⁶ (FIG. 5B) and GSE12093+GSE6532 using GOBO²⁷ (FIG. 5C). The results indicate that increased expression of IL-3 in patients with the “basal” type of cancer have a poorer prognosis in “overall survival” compared to those with low levels of IL-3 (FIG. 5A). The results also indicate that increased expression of IL-3 correlates with poor relapse-free survival (FIG. 5B) and metastasis-free survival (FIG. 5C).

Blocking IL-3R Prevents VM and Reduces Breast Cancer Progression In Vivo

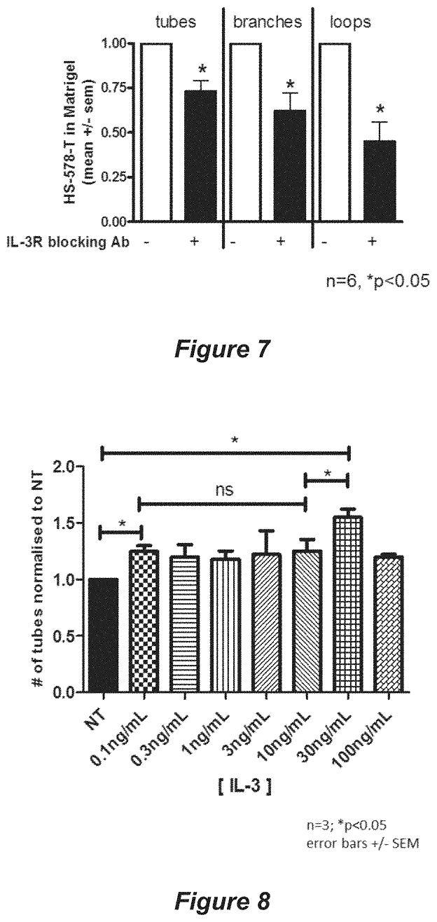

The effect of blocking the IL-3R on the VM capability of MDA-MB-231 cells in Matrigel was analysed. Briefly, an in vitro Matrigel tube forming assay was conducted using MDA-MB-231 cells in combination with a blocking antibody to the IL-3Rα (7G3)¹⁴ (available from BD Pharmingen Inc., San Diego, Calif., United States of America, and shown not to have toxicity in vivo¹⁹). Untreated (NT), IgG or 7G3 were added prior to cell seeding in Matrigel (n=5, *p<0.05). The results showed that the addition of 7G3 significantly attenuated the VM capability of MDA-MB-231 in Matrigel (FIG. 6). The experiment was then repeated with a second human VM competent IDC cell line, HS-578-T, as well as attenuation using a blocking antibody to the IL-3Rβ (BION-1²²; ATCC HB-12525) (FIG. 7), and augmentation with addition of IL-3 (FIG. 8). The results of this experiment supported those shown in FIG. 6.

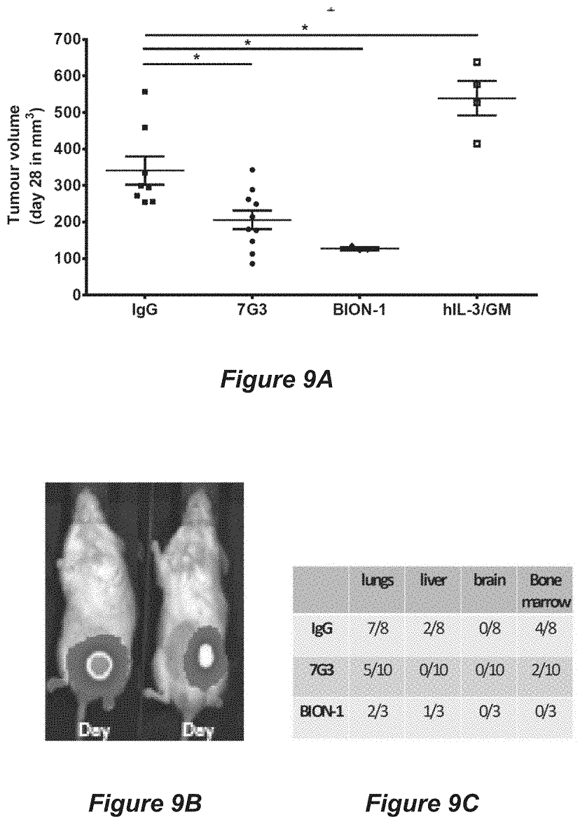

Experiments were conducted in vivo to identify a role for IL-3 in breast cancer progression using modified MDA-MB-231 cells which contain a luciferase tag (MDA-MB-231-LM2)¹⁸. The MDA-MB-231-LM2 cells were implanted (1×10⁶ cells) into the mammary fat pad of 6-week-old NOD/SCID mice and cancer progression monitored using luciferin and bioluminescent imaging with the IVIS imaging system (Xenogen Corporation, Alameda, Calif., United States of America). Seven days post-injection, IgG or anti-7G3 were added every 48 h to the mice. The results showed that intraperitoneal injection of 7G3 (anti-IL-3Rα) or BION-1 (anti-IL-3Rβ) attenuated tumour development in vivo (FIG. 9A).

Mice that were transgenic for the expression of human IL-3/GM-CSF exhibited increased tumour growth. n>4, *p<0.05 vs IgG (FIG. 9A). In FIG. 9B, a representative IVIS image is shown for MDA-MB-231 cells in NOD/SCID mice treated with the control IgG (left) or anti-IL-3Rα (7G3) antibody (right). Investigation of tumour metastasis also indicated that a reduction in IL-3/IL-3R function reduced metastasis to the lung, liver, brain and bone marrow (FIG. 9C). This and data that IDCs express the IL-3R (FIG. 3), produce IL-3 (˜15 μg/ml cell lysate) and bind IL-3 with high affinity, support the notion that the IL-3/IL-3R system plays a significant role in IDC progression.

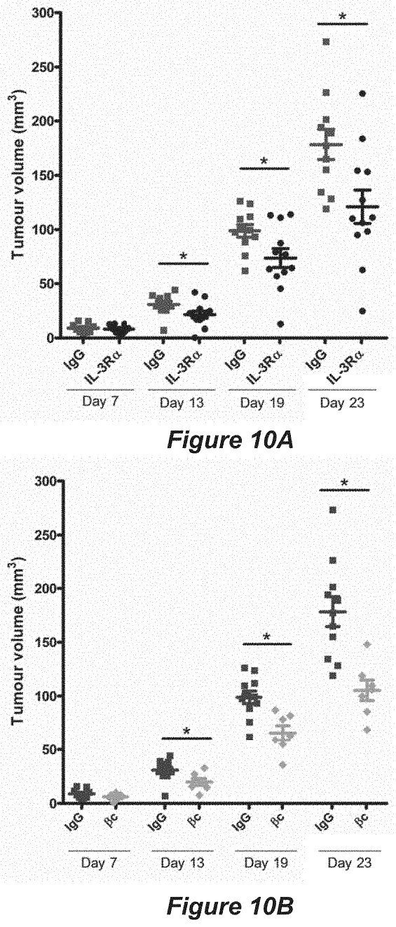

To determine if blocking IL-3Rα or βc could attenuate tumour growth in vivo, 1×10⁶ MDA-MB-231-LM2 cells mixed with Matrigel (1:1 ratio) were subcutaneously injected into the mammary fat pad of 6-8 week old female NOD/SCID mice. Treatments were then by intraperitoneal injection every 2 days once a palpable tumour formed. Tumour growth was monitored via caliper measurements and imaging using a Xenogen IVIS 100 imaging system (Perkin Elmer, Waltham, Mass., United States of America). The results show that blocking the IL-3 receptor with either IL-3Rα (FIG. 10A) or βc (FIG. 10B) in a primary human breast cancer in vivo mouse model attenuated IDC progression by ˜30%. This provides further evidence that an antibody targeting IL-3Rα or βc can be used as a targeted therapeutic for the treatment of breast cancer.

MDA-MB-231-LM2 Cells Unregulate Vascular/Endothelial Cell-Type Genes when Grown In Vivo

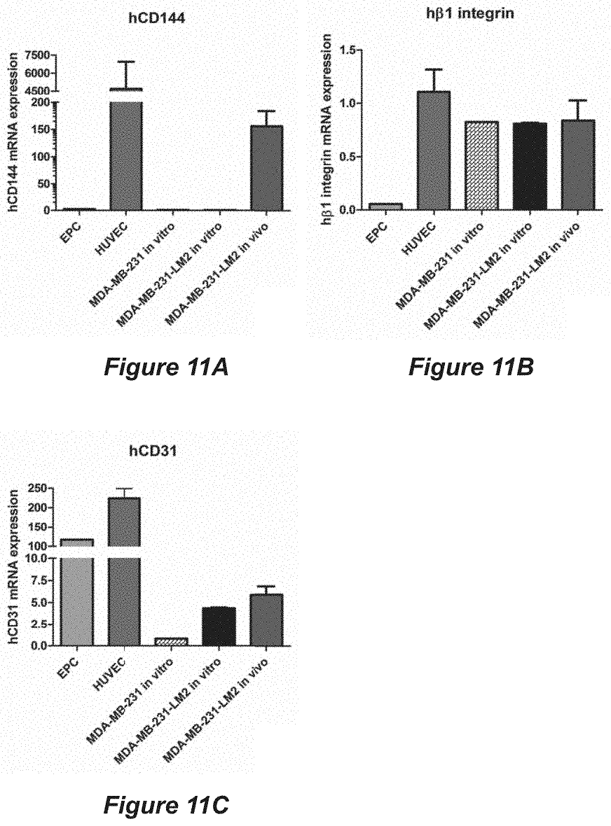

To determine if MDA-MB-231-LM2 cells upregulate vascular/endothelial cell-type genes when grown in vivo, RNA was isolated from tumours extracted from mice (ie tumours from FIG. 10), MDA-MB-231-LM2 cells grown on tissue culture plastic, as well as HUVEC and EPCs. Tumour RNA was extracted by homogenisation with Trizol, then was purified with Qiagen RNeasy Mini kit (Qiagen, Hilden, Germany). RNA was reverse transcribed to cDNA. Quantification of mRNA levels was carried out using qPCR. qPCR amplification was performed using QuantiTect™ SYBR Green master mix (Qiagen) on a Rotor-Gene thermocycler (Qiagen) with reaction parameters: 15 minutes at 95° C., then cycling of 10 seconds 95° C., 20 seconds 55° C. and 30 seconds 72° C.; for 45 cycles followed by a melt phase. Data obtained was analysed using Rotor-Gene Analysis Software version 6 (Qiagen). Relative gene expression levels were calculated using geNorm software by normalising gene expression to the human house-keeping genes cyclophillin A (CycA), GAPDH, and βactin. FIGS. 11A and 11C show that there was an increase mRNA levels of the endothelial cell-type genes CD144 and CD31 in the MDA-MB-231-LM2 tumours excised from the mice. FIG. 11B is a control showing that not all genes involved in vascular development are upregulated (eg β1-integrin). This data shows that when exposed to the tumour microenvironment, tumour cells upregulate vascular marker genes indicating they may be adapting in order to produce vasculogenic mimicry structures.

Mammary Fat Pad Xenografts Produce Human IL-3

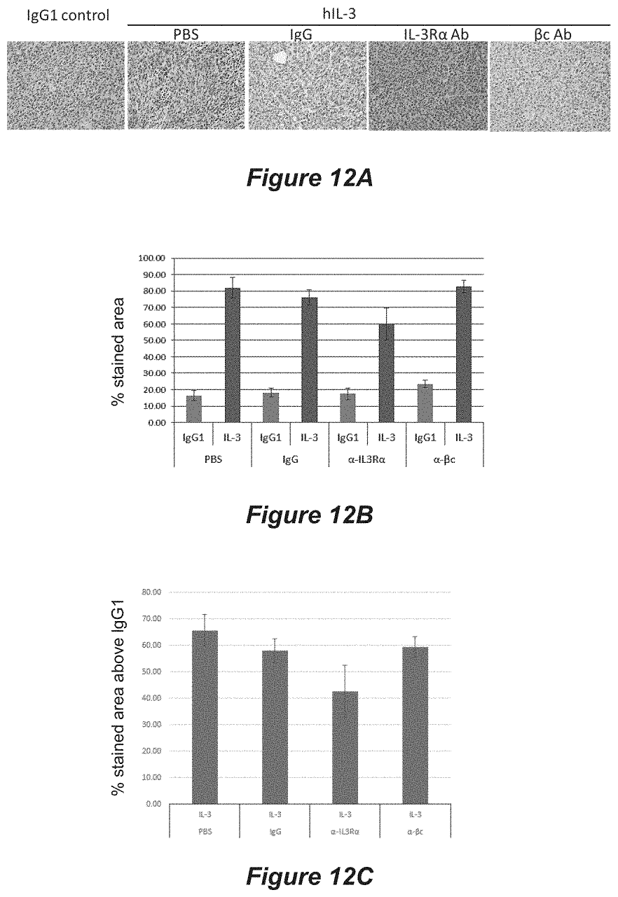

To determine if the human breast cancer cell line MDA-MB-231-LM2 is able to produce human IL-3 (hIL-3) in vivo, primary tumours were excised from mice, washed in 1×PBS, formalin fixed prior to paraffin embedding and then 4 μm sections were cut from these blocks. The staining protocol was as follows: slides were dewaxed then antigen-retrieval was performed using citrate buffer. Tissue was then blocked with 10% normal goat serum, before being incubated with the primary antibody anti-hIL-3 (clone 3B11, GeneTex cat #GTX84295; GeneTex, Inc., Irvine, Calif., United States of America) overnight at 4° C. Slides were then probed with a goat anti-mouse biotin antibody for 1 hour at room temperature. Tissue was then treated with Vectastain ABC kit, then DAB peroxidase substrate kit as per manufacturer's instructions (both Vector Laboratories cat #PK-4000 & SK-4100; Vecor Laboratories, Inc., Burlingame, Calif., United States of America). Slides were then imaged using a Hamamatsu NanoZoomer before being analysed using the online freeware ImmunoRatio (http://153.1.200.58:8080/immunoratio/?locale=en). FIG. 12A shows representative images of tumours extracted from mice treated with PBS, IgG control antibody (IgG), IL-3Rα blocking antibody (IL-3Rα), or βc blocking antibody (βc). FIG. 12B is a graph of compiled data showing the mean percentage area stained positive for IL-3 from 10 fov/tumour±SEM; n=5=6. FIG. 12C is a graph showing IL-3 expression normalised to IgG1 isotype control. The results show that the human cancers grown in these mice are producing IL-3 and that blocking IL-3Rα reduces IL-3 production.

Hypoxia Upregulates IL-3 Receptor Expression on Human Breast Cancer Cell Lines

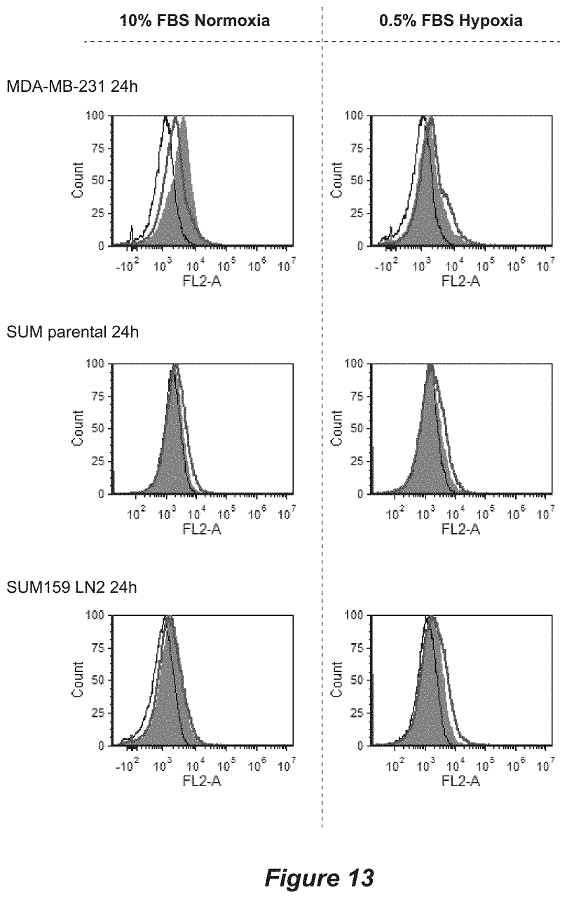

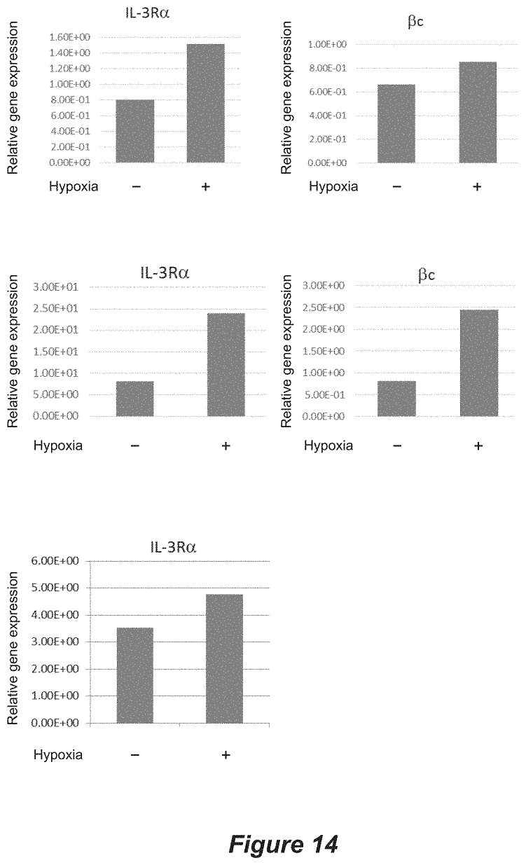

To determine if IL-3 receptor expression is upregulated under conditions of hypoxia MDA-MB-231, SUM159, and SUM159-LN2 breast cancer cell lines were analysed for cell surface expression of IL-3Rα by flow cytometry. Where indicated, cells grown under normal conditions were grown in DMEM with 10% FBS under normal atmospheric oxygen conditions (˜21%), whereas cells grown under hypoxic conditions where grown in DMEM+0.5% FBS in a hypoxia chamber filled with a gas mix containing 3% O₂ for 24 hours prior to harvest. Cells were treated with Human Ig to block Fc receptors prior to the addition of primary antibodies. Primary antibodies were: anti-CD123-PE, and IgG2a-PE (both BD Biosciences, Franklin Fakes, N.J., United States of America) used as per manufacturer's instructions for flow cytometry in a final volume of 50 μL of DMEM+0.5% FBS. 7-AAD was also added prior to fixation. Cells were resuspended in FACS fix (1% formaldehyde, 20 g/L glucose, 5 mM sodium azide in PBS) prior to analysis using an Accuri flow cytometer (BD Biosciences). Further analysis was performed using FCS Express 4 Flow Cytometry: Research Edition (De Novo Software, Glendale, Calif., USA). qPCR was used to analyse gene expression of IL-3Rα and βcommon according to the method used for FIG. 11. The results show that the VM+ breast cancer cells (MDA-MB-231 and SUM159 cells (parental and FN2 lines)) increase their surface expression of IL-3Rα (FIG. 13) as well as gene expression of IL-3Rα and βc when grown under hypoxic conditions (FIG. 14). This indicates that cancer cells (ie tumours) when exposed to environmental stress (eg a hypoxic environment) will upregulate the production/expression of IL-3R subunits.

The present inventors have identified a single unifying factor (IL-3) which appears to control all populations of breast cancer cells with vascular potential (eg VM competent breast cancer cells). In doing so, a new pathogenic marker of breast cancer has been identified that may be targeted in novel therapeutic approaches to treat breast cancers including, potentially, the most aggressive and difficult to manage breast cancers, namely IDCs.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgement of any form of suggestion that such prior art forms part of the common general knowledge.

It will be appreciated by those skilled in the art that the disclosure is not restricted in its use to the particular application described. Neither is the present disclosure restricted in its preferred embodiment with regard to the particular elements and/or features described or depicted herein. It will be appreciated that the disclosure is not limited to the embodiment or embodiments disclosed, but is capable of numerous rearrangements, modifications and substitutions without departing from the scope of the disclosure as set forth and defined by the following claims.

### REFERENCES

1. Alderton G K., Nat Rev Cancer 14(3): 155, 2014.
2. Bender R J and F Mac Gabhann. PLoS One 8(5):e61788, 2013.
3. Ribeiro-Silva A et al., Am J Clin Pathol 125(4):512-518, 2006.
4. Fakhrejahani E and M Toi. Jpn J Clin Oncol 2014.
5. Xu Y et al., J Exp Clin Cancer Res 31:16, 2012.
6. Bertolini F et al., N Engl J Med 353(24):2613-2616, 2005.
7. Mund J A and J Case. Curr Stem Cell Res Ther 6(2): 115-121, 2011.
8. Broughton S et al., Immunol Rev 250:277-302, 2012.
9. Korpelainen et al., Proc Natl Acad Sci USA 90:11137, 1993.
10. Dentelli F et al., J Immunol 163(4):2151-2159, 1999.
11. Sen S et al., Clinical Science 120(7):263, 2011.
12. Appleby S et al., PloS ONE 7:e46996, 2012.
13. Hendrix M J et al., Breast Cancer Res 2(6):417-422, 2000.
14. Sun Q et al., Blood 87:83, 1996.
15. Zeoli A et al., Blood 112(2):350-361, 2008.
16. D'Atri L P et al., Transfusion 51(8): 1784-1795, 2011.
17. Finak G et al., Nat Med 14(5):518-527, 2008.
18. Minn A J et al., Nature 436(7050):518-524, 2005.
19. Jin L et al., Cell Stem Cell 5:31-42, 2009.
20. Busfield S et al., Leukaemia 28(11):2213-2221, 2014.
21. Gluck S et al., Breast Cancer Res Treat 2011.
22. Ramshaw H S et al., Immun Cell Biol 79:154-159, 2001.
23. Foulkes W D et al., N Eng J Med 363(20): 1938-1948, 2010.
24. Ebos J M et al., Cancer Cell 15(3):232-239, 2009.
25. Gyorffy B et al., Breast Cancer Res Treat 123(3):725-731, 2010.
26. Buffa F M et al., Cancer Res 71(17):5635-5645, 2011.
27. Ringner M et al., PLoS One. 6(3):e17911, 2011.
28. Lopez A F et al, IUBMB Life 62(7):509-518, 2010.
29. Shoemaker, S G et al., PNAS 87: 9650-9654, 1990.
30. Jaster, R et al., Cell Signal 11(10):769-775, 1999.
31. Nolan J P and L A Sklar. Trends Biotechnol 20(1):9-12 (2002).
32. Vandesompele J et al., Genome Biology 3(7): 1-12 (2002).
33. The Cancer Genome Atlas Research Network, Nature 499(7456):43-49 (2013).
34. Nutt C L et al., Cancer Res. 63(7): 1602-1607 (2003).
35. Mischel P et al., Oncogene. 22(15):2361-2373 (2003).
36. Bhattacharjee A, Proc Natl Acad Sci USA 98(24): 13790-13795 (2001)
37. The Cancer Genome Atlas Research Network Nature 489(7417):519-525 (2012).
38. Mroczko B et al., Przegl Lek. 56(12):763-766 (1999).

1. A method of treating or preventing breast cancer in a subject, said method comprising administering to said subject an interleukin-3 (IL-3)-inhibiting agent selected from inhibitory anti-IL3Rα antibodies and IL3Rα-binding fragments thereof and inhibitory anti-IL3Rβ antibodies and IL3Rβ-binding fragments thereof:
wherein the breast cancer is associated with elevated levels of IL-3 in the subject; and wherein the breast cancer is negative for oestrogen receptors (ER−), progesterone receptors (PR−) and HER2 (HER2−).

2. The method of claim 1, wherein the breast cancer is an invasive ductal carcinoma (IDC).

3. (canceled)

4. (canceled)

5. The method of claim 1, wherein the breast cancer is considered as having vascular potential.

6. The method of claim 1, wherein the breast cancer of the subject has vascular potential or is vasculogenic mimicry (VM) competent.

7. The method of claim 1, wherein the IL-3-inhibiting agent inhibits the activity of endogenous IL-3 in the subject.

8.-13. (canceled)

14. The method of claim 1, wherein the IL-3-inhibiting agent is administered simultaneously or sequentially with one or more additional agent(s) for the treatment of breast cancer.

15.-26. (canceled)

27. A method for the treatment of metastasis in a subject suffering from breast cancer, said method comprising administering to said subject an IL-3-inhibiting agent selected from inhibitory anti-IL3Rα antibodies and IL3Rα-binding fragments thereof and inhibitory anti-IL3Rβ antibodies and IL3Rβ-binding fragments thereof;
wherein the breast cancer is associated with elevated levels of IL-3 in the subject; and
wherein the breast cancer is negative for oestrogen receptors (ER⁻) progesterone receptors (PR⁻) and HER2 (HER2⁻).

28.-62. (canceled)

63. The method of claim 27, wherein the breast cancer is an invasive ductal carcinoma (IDC).

64. The method of claim 27, wherein the breast cancer is considered as having vascular potential.

65. The method of claim 27, wherein the breast cancer of the subject has vascular potential or is vasculogenic mimicry (VM) competent.

66. The method of claim 27, wherein the IL-3-inhibiting agent inhibits the activity of endogenous IL-3 in the subject.

67. The method of claim 27, wherein the IL-3-inhibiting agent is administered simultaneously or sequentially with one or more additional agent(s) for the treatment of breast cancer.
